(19) United States
(12) Patent Application Publication
(10) Pub. No.: US 2026/0022120 A1
(43) Pub. Date: Jan. 22, 2026

(54) Inhibitors of MENIN-MLL Interaction

(71) Applicant: Bala Therapeutics, Inc., Dover, DE (US)

(72) Inventors: Ruben Abagyan, La Jolla, CA (US); Vladislav Zenonovich Parchinsky, Moscow (RU); Nikolay Savchuk, San Diego, CA (US); Alexandre Vasilievich Ivachtchenko, Hallandale Beach, FL (US); Alexander Khvat, San Diego, CA (US)

(21) Appl. No.: 19/334,801

(22) Filed: Sep. 19, 2025

Related U.S. Application Data
(62) Division of application No. 18/717,973.
(60) Provisional application No. 63/287,716, filed on Dec. 9, 2021.
(60) Provisional application No. 63/306,399, filed on Feb. 3, 2022.
(60) Provisional application No. 63/397,322, filed on Aug. 11, 2022.

(51) Int. Cl. C07D 471/10; A61K 31/5377; A61K 31/553; A61K 45/06; C07D 519/00

(52) U.S. Cl. CPC C07D 471/10; A61K 31/5377; A61K 31/553; A61K 45/06; C07D 519/00

(57) ABSTRACT

The present invention is directed to the compounds 101 and 102—inhibitors of the interaction of menin and MLL. The inhibitors described herein can be useful in the treatment of diseases or disorders associated with menin-MLL interaction, such as cancer. In particular, the invention is concerned with compounds and pharmaceutical compositions inhibiting/blocking menin-MLL interaction, methods of treating diseases or disorders associated with menin-MLL interaction, and methods of synthesizing these compounds.

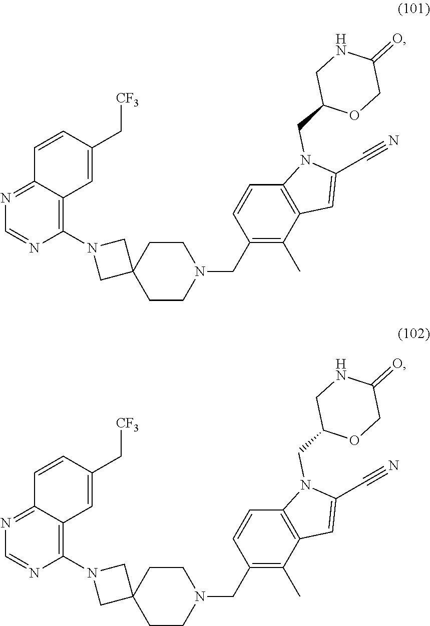 [SMILES: Cc1c(CN2CCC3(CC2)CN(c2ncnc4ccc(CC(F)(F)F)cc24)C3)ccc2c1cc(C#N)n2C[C@H]1CNC(=O)CO1 ; Cc1c(CN2CCC3(CC2)CN(c2ncnc4ccc(CC(F)(F)F)cc24)C3)ccc2c1cc(C#N)n2C[C@@H]1CNC(=O)CO1]

### CROSS-REFERENCE TO RELATED APPLICATIONS

This application is division application of U.S. Utility patent application Ser. No. 18/717,973, which claims priority to, and the benefit of, U.S. Provisional Patent Application Ser. No. 63/287,716 filed on Dec. 9, 2021 and entitled “INHIBITORS OF MENIN-MLL INTERACTION,” U.S. Provisional Patent Application Ser. No. 63/306,399 filed on Feb. 3, 2022 and entitled “INHIBITORS OF MENIN-MLL INTERACTION,” and U.S. Provisional Patent Application Ser. No. 63/397,322 filed on Aug. 11, 2022 and entitled “INHIBITORS OF MENIN-MLL INTERACTION,” the disclosures of which are incorporated herein by reference in their entireties for all purposes.

### FIELD OF INVENTION

The present invention is directed to inhibitors of the interaction of menin and MLL. The inhibitors described herein can be useful in the treatment of diseases or disorders associated with menin-MLL interaction, such as cancer. In particular, the invention is concerned with compounds and pharmaceutical compositions inhibiting/blocking menin-MLL interaction, methods of treating diseases or disorders associated with menin-MLL interaction, and methods of synthesizing these compounds.

### BACKGROUND

Translocations of the MLL (mixed lineage leukemia) gene frequently occur in aggressive human acute myeloid and lymphoid leukemias in both children and adults. Fusion of MLL with 1 of more than 60 different genes results in chimeric MLL fusion proteins that enhance proliferation and block hematopoietic differentiation, ultimately leading to acute leukemia. Patients with leukemias harboring MLL translocations have very unfavorable prognoses and respond poorly to currently available treatments. The relapse risk is very high using conventional chemotherapy and stem cell transplantation, leading to an overall 5-year survival rate of only approximately 35% of patients.

Menin is an essential co-factor of oncogenic MLL fusion proteins and the menin-MLL interaction is critical for development of acute leukemia in vivo. Targeting the menin-MLL interaction with small molecules represents an attractive strategy to develop new anticancer agents. Recent developments, including determination of menin crystal structure and development of potent small molecule and peptidomimetic inhibitors, demonstrate feasibility of targeting the menin-MLL interaction. On the other hand, biochemical and structural studies revealed that MLL binds to menin in a complex bivalent mode engaging two MLL motifs, and therefore inhibition of this protein-protein interaction represents a challenge.

Chromosomal rearrangements of the MLL gene located at chromosome band 11q23 are found in patients with de novo acute myeloid (AML) and acute lymphoblastic (ALL) leukemias, and in therapy related leukemias or myelodysplastic syndrome (MDS). As a consequence of chromosomal translocations, the MLL gene is fused with one of over 60 different protein partners, such as the most frequent AF4, AF9, ENL, AF6, ELL, and AF10. Disruption of MLL by gene fusions upregulates expression of HOXA9 and MEIS1 genes that are critical to leukemogenesis. The role of HOXA genes in leukemic transformation has been verified in both, in vitro and in vivo models, demonstrating that MLL fusion protein mediated upregulation of HOXA9 and MEIS1 genes results in enhanced proliferation and blockage of hematopoietic differentiation, ultimately leading to acute leukemia. Patients with leukemias harboring MLL translocations have very unfavorable prognosis (20% event free survival at 3 years) and respond poorly to available treatments, demonstrating a clear need for new therapies.

The oncogenic function of MLL fusion proteins is critically dependent on their direct interaction with menin. Menin is a 67 kDa protein encoded by the MEN1 (Multiple Endocrine Neoplasia I) gene localized on chromosome 11q13. Menin is a ubiquitously expressed protein, predominantly localized in the nucleus. Menin directly binds to the N-terminus of MLL that is retained in all MLL fusion proteins and plays an important role in recruitment of MLL and MLL fusions to target genes, including HOXA9. Loss of menin binding by MLL fusion proteins abolishes their oncogenic properties in vitro and in vivo. Mutations within the N-terminus of MLL-ENL oncoprotein, resulting in protein unable to associate with menin, abolish its potential to upregulate Hox gene expression and induce leukemia in mice. Expression of a dominant-negative inhibitor composed of the amino terminal MLL sequence inhibits growth of the MLL-AF9 transformed bone marrow cells and blocks leukemogenic transformation.

Inhibiting the interaction of menin with the histone methyltransferase MLL1 (KMT2A) has recently emerged as a novel therapeutic strategy. Beneficial therapeutic effects have been postulated in leukemia, prostate, breast, liver and in synovial sarcoma models. In those indications, MILLI recruitment by menin was described to critically regulate the expression of disease associated genes.

Blocking the menin-MLL interaction might represent a viable approach to reverse the oncogenic activity of MLL fusion proteins in leukemia and may lead to novel therapeutics.

### SUMMARY

A first aspect of the invention relates to compounds 101 and 102:

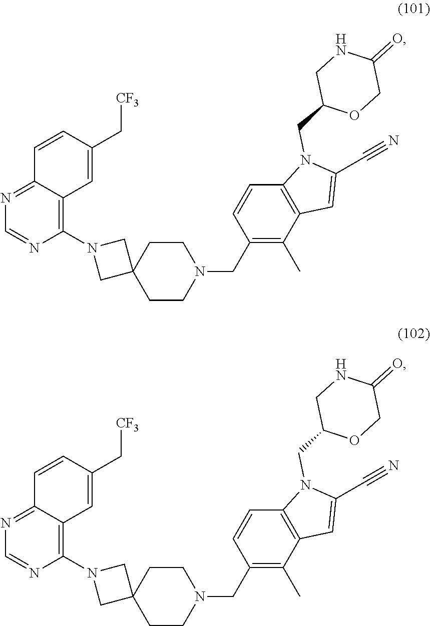 [SMILES: Cc1c(CN2CCC3(CC2)CN(c2ncnc4ccc(CC(F)(F)F)cc24)C3)ccc2c1cc(C#N)n2C[C@H]1CNC(=O)CO1 ; Cc1c(CN2CCC3(CC2)CN(c2ncnc4ccc(CC(F)(F)F)cc24)C3)ccc2c1cc(C#N)n2C[C@@H]1CNC(=O)CO1]

or a pharmaceutically acceptable salt, prodrug, stereoisomer, solvate, or tautomer thereof.

Another aspect of the invention is directed to pharmaceutical compositions comprising compound 101 or/and 102, or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with interaction of menin and MLL. The method comprises administering to a patient in need of a treatment for diseases or disorders associated with interaction of menin and MLL an effective amount of compound 101 or/and 102, or a pharmaceutically acceptable salt, hydrate, solvate, prodrug, stereoisomer, tautomer, or pharmaceutical composition thereof.

Another aspect of the invention is directed to a method of inhibiting of interaction of menin and MLL. The method involves administering to a patient in need thereof an effective amount of compound 101 or/and 102, or a pharmaceutically acceptable salt, hydrate, solvate, prodrug, stereoisomer, tautomer, or pharmaceutical composition thereof.

Another aspect of the present invention relates to compounds 101 or/and 102, or pharmaceutically acceptable salts, hydrates, solvates, prodrugs, stereoisomers, tautomers, or pharmaceutical compositions thereof, for use in the manufacture of a medicament for inhibiting interaction of menin and MLL.

Another aspect of the present invention relates to the use of compound 101 or/and 102, or pharmaceutically acceptable salts, hydrates, solvates, prodrugs, stereoisomers, tautomers, or pharmaceutical compositions thereof, in the treatment of a disease associated with inhibiting of interaction of menin and MLL.

Another aspect of the present invention relates to compound 101 or/and 102, or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound 101 or/and 102, or a pharmaceutically acceptable salt, hydrate, solvate, prodrug, stereoisomer, tautomer, or pharmaceutical composition thereof.

Another aspect of the present invention relates to the use of compound 101 or/and 102, or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with interaction of menin and MLL, comprising administering to a patient suffering from at least one of said diseases or disorders compound 101 or/and 102, or a pharmaceutically acceptable salt, hydrate, solvate, prodrug, stereoisomer, tautomer, or pharmaceutical composition thereof.

The present invention provides inhibitors of interaction of menin and MLL that are therapeutic agents in the treatment of diseases and disorders.

The present invention further provides compounds and compositions with an improved efficacy and safety profile relative to known inhibitors of menin and MLL interaction. The present disclosure also provides agents with novel mechanisms of action toward interaction of menin and MLL in the treatment of various types of diseases.

The present invention further provides methods of treating a disease or disorder associated with interaction of menin and MLL, comprising administering to a patient suffering from at least one of said diseases or disorders compound 101 or/and 102, or a pharmaceutically acceptable salt, hydrate, solvate, prodrug, stereoisomer, tautomer, or pharmaceutical composition thereof.

The present invention provides inhibitors of interaction of menin and MLL that are therapeutic agents in the treatment of diseases and disorders.

The present invention further provides methods of treating a disease, disorder, or condition selected from cancer, acute myeloid (AML) and acute lymphoblastic (ALL) leukemias, or myelodysplastic syndrome (MDS), comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a disease or disorder in which associated with the inhibition of the interaction of menin and MLL1 by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compound 101 or/and 102 may have one or more asymmetric carbon atom and may occur as racemates, racemic mixtures and as individual enantiomers or diastereomers.

The present disclosure also contemplates isotopically-labelled compound 101 or/and 102 (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compound 101 or/and 102 can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound The term “salt’ refers to pharmaceutically acceptable salts

The term “pharmaceutically acceptable salt” also refers to a salt of the compositions of the present disclosure having an acidic functional group, such as a carboxylic acid functional group, and a base.

“Menin/MLL interaction inhibitor” as used herein refer to compound 101 or/and 102 and/or compositions comprising compound 101 or/and 102 which inhibits the interaction of menin and MLL.

The amount of compound of composition described herein needed for achieving a therapeutic effect may be determined empirically in accordance with conventional procedures for the particular purpose. Generally, for administering therapeutic agents (e.g. compounds or compositions disclosed herein (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

### Compounds of the Present Disclosure

In one aspect, the present disclosure provides compound 101 or/and 102 and salts, stereoisomers, solvates, prodrugs, isotopic derivatives, and tautomers thereof.

In some embodiments, the compound is a neutral form (i.e., not a salt) of compound 101 or/and 102.

In some embodiments, the compound is a pharmaceutically acceptable salt of compound 101 or/and 102.

In some aspects, the present disclosure provides a compound being an isotopic derivative (e.g., isotopically labeled compound) of compound 101 or/and 102.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

The term “isotopic derivative”, as used herein, refers to a derivative of a compound in which one or more atoms are isotopically enriched or labelled. For example, an isotopic derivative of compound 101 or/and 102 is isotopically enriched with regard to, or labelled with, one or more isotopes as compared to the corresponding compound 101 or/and 102. In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof).

In some embodiments, the compound is a deuterium labeled compound.

It will be understood that the compound 101 or/and 102 and any pharmaceutically acceptable salts thereof, comprise stereoisomers, mixtures of stereoisomers, polymorphs of all isomeric forms of said compounds.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

It is to be understood that the compound 101 or/and 102 include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The compounds disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound disclosed herein may be a synthetically produced compound or a metabolically-produced compound.

A suitable pharmaceutically acceptable prodrug of a compound disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

The in vivo effects of a compound disclosed herein may be exerted in part by one or more metabolites that are formed within the human or animal body after administration of a compound disclosed herein. As stated hereinbefore, the in vivo effects of a compound disclosed herein may also be exerted by way of metabolism of a precursor compound (a prodrug).

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound described herein, or a pharmaceutically acceptable salt or solvate thereof, and one or more pharmaceutically acceptable carriers or excipients.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated B-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-y-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ¿-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a MLL related condition referred to herein, slow its progression and/or reduce the symptoms associated with the condition.

A therapeutically effective amount of a compound of the present disclosure for use in therapy is an amount sufficient to treat an MLL related condition referred to herein, slow its progression and/or reduce the symptoms associated with the condition.

The size of the dose for therapeutic or prophylactic purposes of compound 101 or/and 102 will naturally vary according to the nature and severity of the conditions, the age and sex of the animal or subject and the route of administration, according to well-known principles of medicine.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Starting materials are either commercially available or made by known procedures in the reported literature or as illustrated below.

### Biological Assays

Compounds disclosed herein can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Methods of Use

In some aspects, the present disclosure provides a method of inhibiting the interaction of menin with MLL (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with MLL. In some embodiments, the disease or disorder is a disease or disorder in which menin-MLL binding is implicated.

The compounds of the invention are inhibitors of the interaction of menin with MLL and MLL fusion proteins. In some embodiments, the present invention is directed to a method of inhibiting the interaction between menin and MLL or an MLL fusion protein by contacting menin and MLL or the MLL fusion protein with a compound of the invention. The contacting can be carried out in vitro or in vivo. In some embodiments, the compounds of the invention can bind to menin, thereby interfering with the binding of MLL to menin. In some embodiments, the present invention provides a method of inhibiting the activity of menin by contacting menin with a compound of the invention in the presence of MLL or an MLL fusion protein. In further embodiments, the present invention provides a method of inhibiting the binding of MLL or an MLL fusion protein to menin, comprising contacting menin with a compound of the invention in the presence of the MLL or MLL fusion protein.

The compounds of the invention are also useful in treating diseases associated with the menin-MLL interaction or menin-MLL fusion protein interaction. For example, diseases and conditions treatable according to the methods of the invention include cancer, such as leukemia, and other diseases or disorders mediated by the menin-MLL interaction or menin-MLL fusion protein interaction such as diabetes.

In some embodiments, the disease or disorder is selected from the group consisting of a leukemia, hematologic malignancy, solid tumor cancer, prostate cancer, breast cancer, liver cancer, brain tumor, and diabetes. In some embodiments, the leukemia is selected from the group consisting of AML, ALL, Mixed Lineage Leukemia, and a leukemia with Partial Tandem Duplications of MLL.

In some embodiments, the disease or disorder is a cancer.

In some embodiments, the cancer is selected from hematological cancer (e.g., leukemia and lymphoma), bladder cancer, brain cancer (e.g., glioma), diffuse intrinsic pontine glioma (DIPG)), breast cancer (e.g., triple-negative breast cancer), colorectal cancer, cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer, renal cancer (e.g., renal cell carcinoma), skin cancer, thyroid cancer (e.g., papillary thyroid carcinoma), testicular cancer, sarcoma (e.g., Ewing's sarcoma), and AIDS-related cancers. In some embodiments, cancer is selected from cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g., adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer, extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodeimal tumors, pineoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenstrom's macroglobulinemia), and other lymphatic or hematologic cancers including, for example, childhood leukemia, myeloproliferative disorders (e.g., primary myelofibrosis), plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Sezary Syndrome;

skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor; adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors); connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukemia or lymphoma. Example leukemia and lymphomas treatable by the compounds of the invention include mixed lineage leukemia (MLL), MLL-related leukemia, MLL-associated leukemia, MLL-positive leukemia, MLL-induced leukemia, rearranged mixed lineage leukemia (MLL-r), leukemia associated with a MLL rearrangement or a rearrangement of the LJ gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non-Hodgkin's lymphoma (malignant lymphoma), and Waldenstrom's macroglobulinemia.

In some embodiments, diseases and conditions treatable with compounds of the invention include insulin resistance, pre-diabetes, diabetes (e.g., Type 2 diabetes or Type 1 diabetes), and risk of diabetes. In some embodiments, diseases and conditions treatable with compounds of the invention include hyperglycemia. In some embodiments, the hyperglycemia is associated with diabetes, such as Type 2 diabetes. In some embodiments, compounds of the invention are used to treat loss of response to other anti-diabetic agents and/or reduced beta cell function in a patient or subject. In some embodiments, compounds of the invention are used to restore response to other anti-diabetic agents and/or to restore beta cell function and/or to reduce the need for insulin in a patient or subject. In some embodiments, compounds of the invention are used to reduce insulin resistance, reduce the risk of diabetes, or reduce increases in blood glucose caused by a statin in a subject taking a statin. In some embodiments, compounds of the invention are used to treat diabetes in a subject taking a statin or to prevent diabetes in a subject taking a statin. Methods of the invention include decreasing, reducing, inhibiting, suppressing, limiting or controlling in the subject elevated blood glucose levels. In further aspects, methods of the invention include increasing, stimulating, enhancing, promoting, inducing or activating in the subject insulin sensitivity. Statins include, but are not limited to atorvastatin, cerivastatin, fluvastatin, lovastatin, mevastatin, pitavastatin, pravastatin, rosuvastatin and simvastatin.

In some aspects, the present disclosure provides a method of treating or preventing a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting the interaction of menin with MLL (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a cancer in a subject in need thereof.

In some aspects, the present disclosure provides a compound of the present disclosure or a pharmaceutically acceptable salt thereof for use in treating a cancer in a subject in need thereof.

In some aspects, the present disclosure provides use of a compound of the present disclosure or a pharmaceutically acceptable salt thereof in the manufacture of a medicament for inhibiting the interaction of menin with MLL (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a cancer in a subject in need thereof.

In some aspects, the present disclosure provides use of a compound of the present disclosure or a pharmaceutically acceptable salt thereof in the manufacture of a medicament for treating a cancer in a subject in need thereof.

The present disclosure provides compounds that function as inhibitors of the interaction of menin with MLL (e.g., in vitro or in vivo). The present disclosure therefore provides a method of inhibiting the interaction of menin with MLL in vitro or in vivo, said method comprising contacting a cell with a therapeutically effective amount of a compound, or a pharmaceutically acceptable salt thereof, as defined herein.

In some embodiments, the menin/MLL interaction inhibitor is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which interaction of menin with MLL is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Abbreviations used in the following examples and elsewhere herein are:
aq. aqueous
Boc₂O di-tert-butyl dicarbonate
br. broad
BSA bovine serum albumin
d duplet
DCM dichloromethane
DIPEA N,N-diisopropylethylamine
DMAA 1,3-dimethylamylamine
DMAP 4-(dimethylamino)pyridine
DMEM Dulbecco's modified Eagle's medium
DMF N,N-dimethyl formamide
DMSO dimethyl sulfoxide
DTT dithiothreitol
FBS fetal bovine serum
h hour(s)
LCMS liquid chromatography mass spectrometry
m multiplet
M molar
MHz megahertz
min minutes
NBS N-bromosuccinimide
NMR nuclear magnetic resonance
Pd₂(dba)₃ tris (dibenzylideneacetone) dipalladium (0)
Pd(dppf)Cl₂[1,1′-bis(diphenylphosphino) ferrocene]dichloropalladium (II)
q quadruplet
rt room temperature
S singlet
STAB sodium triacetoxyborohydride
t temperature, triplet
TEA triethyl amine
THF tetrahydrofuran
TLC thin layer chromatography
TRIS 2-amino-2-(hydroximethyl) propane-1,3-diol

### EXAMPLES

### General Synthetical Procedures and Examples of the Compound's Preparation

### Synthesis of Building Blocks

### Synthesis of 4-chloro-6-(2,2,2-trifluoroethyl)quinazoline (P7)

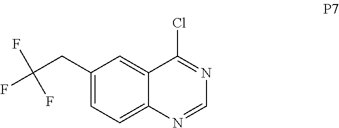 [SMILES: FC(F)(F)Cc1ccc2ncnc(Cl)c2c1]

### Preparation 1. Methyl 5-bromo-2-[(tert-butoxycarbonyl) amino]benzoate (P1)

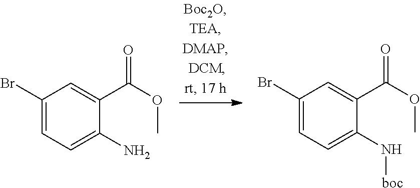

To solution of methyl 2-amino-5-bromobenzoate (25 g, 0.109 mol) in DCM (250 ml) was added Boc₂O (26.5 g, 0.122 mol), triethylamine (62 g, 0.61 mol) and DMAP (4 g, 0.033 mol). The solution was stirred at ambient temperature for 17 h. Then 250 ml of water was added, and potassium hydrosulfate was added in small portions with stirring until pH reached 3. Water phase was washed with dichloromethane (200 ml), organic solution was concentrated, and residue was purified using silica gel column chromatography (30% dichloromethane in hexane). 5-Bromo-2-[(tert-butoxycarbonyl) amino]benzoate (P1) was isolated as white solid (14.3 g, 40%). ¹H NMR (400 MHZ, DMSO-d₆), δ: 10.02 (s, 1H), 8.10 (d, J=9.0 Hz, 1H), 7.98 (d, J=2.4 Hz, 1H), 7.76 (d, J=9.0, 2.4 Hz, 1H), 3.84 (s, 3H), 1.43 (s, 9H).

### Preparation 2. Methyl 2-[(tert-butoxycarbonyl)amino]-5-(4,4,5,5-tetramethyl-1,3,2-dioxaborolan-2-yl)benzoate (P2)

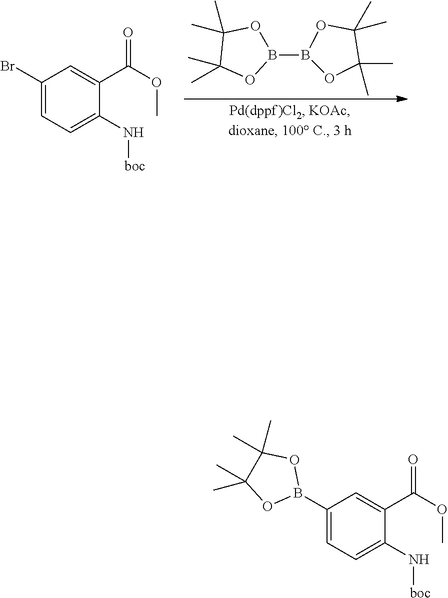

5-Bromo-2-[(tert-butoxycarbonyl) amino]benzoate (P1, 14.3 g, 0.043 mol) and bis (pinacolato)diboron (22 g, 0.087 mol) were dissolved in dioxane (120 ml). Potassium acetate (12.6 g, 0.129 mol) was added to the solution. The mixture was stirred for 30 min at 70° C. under argon atmosphere. Then Pd(dppf)Cl₂ (3.2 g) was added, and reaction mixture was stirred at 100° C. for additional 3 h (TLC control). The mixture was cooled down to rt and filtered through a pad of celite. Filtrate was then concentrated, and residue was purified by silica gel column chromatography (dichloromethane) to give the product (P2) as a white solid (16 g, 99% yield). ¹H NMR (400 MHZ, DMSO-d₆), δ: 10.31 (s, 1H), 8.26 (d, J=8.8 Hz, 2H), 7.83 (d, J=8.4 Hz, 1H), 3.87 (s, 3H), 1.48 (s, 9H), 1.29 (s, 12H).

### Preparation 3 Methyl 2-[(tert-butoxycarbonyl)amino]-5-(2,2,2-trifluoroethyl)benzoate (P3)

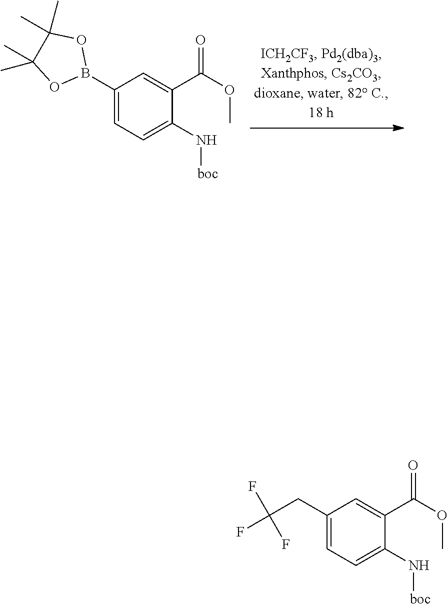

Methyl 2-[(tert-butoxycarbonyl)amino]-5-(4,4,5,5-tetramethyl-1,3,2-dioxaborolan-2-yl)benzoate (P2, 11.2 g, 30 mmol) was dissolved in dioxane (100 ml), cesium carbonate (32.6 g, 118 mmol) in water (8 ml) was added. The mixture was stirred 30 min at 70° C. under argon atmosphere. Then 1,1,1-trifluoro-2-iodo-ethane (18.6 g, 89 mmol), Pd₂(dba)₃ (2.6 g), and Xantphos (2.6 g) were added. The reaction mixture was stirred at 82° C. for 18 h, then the mixture was cooled down and filtered through a pad of celite. The filtrate was concentrated. The residue was purified by silica gel column chromatography (dichloromethane-hexane-1:1) to give the product (P3) as a white solid (5.85 g, 59% yield). ¹H NMR (400 MHZ, DMSO-d₆), δ: 10.10 (s, 1H), 8.20 (t, J=20.8 Hz, 1H), 7.92 (s, 1H), 7.50 (d, J=23.8 Hz, 1H), 3.86 (s, 3H), 3.74-3.48 (m, 2H), 1.48 (s, 9H).

### Preparation 4. Methyl 2-amino-5-(2,2,2-trifluoroethyl)benzoate (P4)

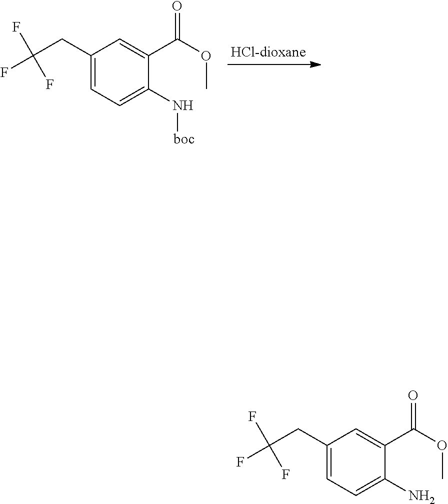

3M Solution of HCl in dioxane was added to methyl 2-[(tert-butoxycarbonyl)amino]-5-(2,2,2-trifluoroethyl)benzoate (P3, 5.85 g, 17.6 mmol). Reaction mixture was stirred at rt for 2 h (NMR control) and concentrated to dryness to give the product (P4, 4.6 g, 100%) as hydrochloride. ¹H NMR (400 MHZ, DMSO-d₆), δ: 7.62-7.70 (m, 1H), 7.19-7.27 (m, 1H), 6.86-6.84 (m, 1H), 3.79 (s, 3H), 4.03-3.58 (m, 2H).

### Preparation 5. 2-Amino-5-(2,2,2-trifluoroethyl)benzoic acid (P5)

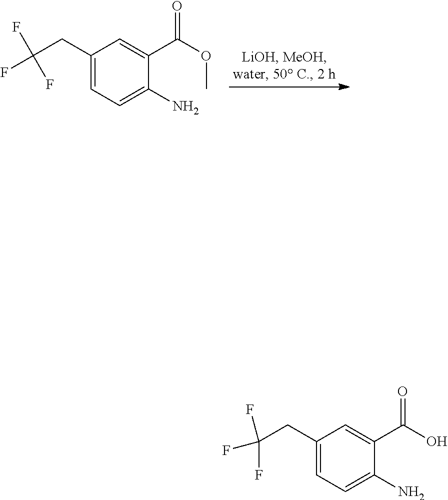 [SMILES: COC(=O)c1cc(CC(F)(F)F)ccc1N ; Nc1ccc(CC(F)(F)F)cc1C(=O)O]

Lithium hydroxide (2 g, 83 mmol) in water (35 ml) was added to the solution of methyl 2-amino-5-(2,2,2-trifluoroethyl)benzoate (P4, 4.6 g, 17.6 mmol) in methanol (100 ml). The reaction mixture was stirred at 50° C. for 2 h. Then reaction mixture was cooled, concentrated to dryness, and water (50 ml) was added. Concentrated HCl was added to the obtained solution with stirring and cooling with cold water up to pH=3. Obtained precipitate was filtered off, washed with water (15 ml) and dried to give the product P5 as a white solid (3 g, 78%). ¹H NMR (400 MHZ, DMSO-d₆), δ: 7.62-7.70 (s, 1H), 7.23 (d, J=8.3 Hz, 1H), 6.81 (d, J=8.1 Hz, 1H), 3.55-3.33 (m, 2H).

### Preparation 6. 6-(2,2,2-Trifluoroethyl)quinazolin-4 (3H)-one (P6)

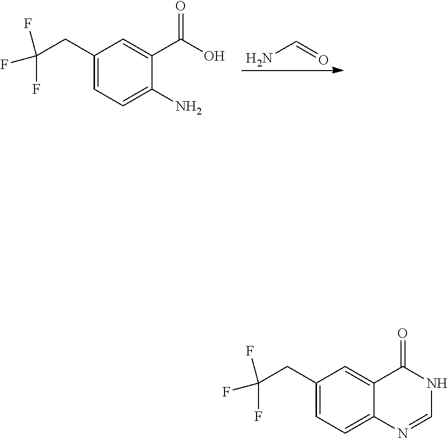

2-Amino-5-(2,2,2-trifluoroethyl)benzoic acid (P5, 3 g, 13.7 mmol) and formamide (2.2 g, 49 mmol) were stirred in vial at 155° C. for 1.5 h, and then at 165° C. for 40 min. The reaction mixture was cooled, saturated solution of sodium bicarbonate (50 ml) was added, and the mixture stirred at rt for 30 min. Formed precipitate was filtered off, washed with water (55 ml) and dried to give the product P6 as a white solid (2.52 g, 85%). ¹H NMR (400 MHZ, DMSO-d₆), δ: 12.30 (s, 1H), 8.4-8.16 (m, 1H), 8.07-8.16 (m, 1H), 7.78 (d, J=8.6 Hz, 1H), 7.68 (d, J=7.8 Hz, 1H), 3.79-3.90 (m, 2H).

### Preparation 7. 4-Chloro-6-(2,2,2-trifluoroethyl)quinazoline (P7)

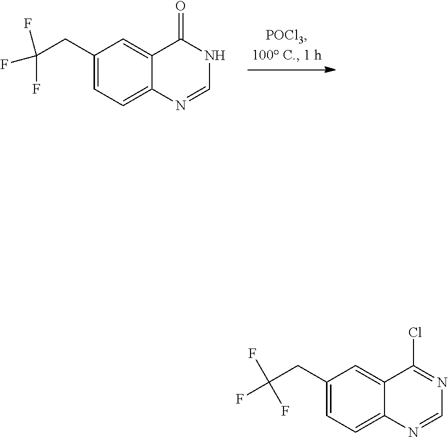 [SMILES: O=c1[nH]cnc2ccc(CC(F)(F)F)cc12 ; FC(F)(F)Cc1ccc2ncnc(Cl)c2c1]

POCl₃ (35 ml) was added to 6-(2,2,2-trifluoroethyl)quinazolin-4 (3H)-one (P6, 2.52 g, 11 mmol), the reaction mixture was stirred and refluxed for 1 h. The solution was cooled down and poured to ice-cold water solution of sodium bicarbonate. The mixture was stirred for 30 min, then extracted with DCM (2×50 ml). After evaporation of dichloromethane, residue was purified by silica gel column chromatography (dichloromethane-ethyl acetate-10:1) to give the product (P7, 1.9 g 70%) as a brown solid. ¹H NMR (400 MHz, CDCl₃), &: 9.12-9.07 (m, 1H), 8.25-8.22 (m, 1H), 8.12 (d, J=8.3 Hz, 1H), 7.93 (d, J=8.1, Hz, 1H), 3.70-3.60 (m, 2H).

### Synthesis of 4-(2,7-diazaspiro[3.5]non-2-yl)-6-(2,2,2-trifluoroethyl)quinazoline (P9)

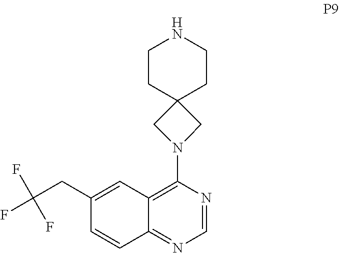 [SMILES: FC(F)(F)Cc1ccc2ncnc(N3CC4(CCNCC4)C3)c2c1]

### Preparation 8. tert-Butyl 2-[6-(2,2,2-trifluoroethyl)quinazolin-4-yl]-2,7-diazaspiro[3.5]nonane-7-carboxylate (P8)

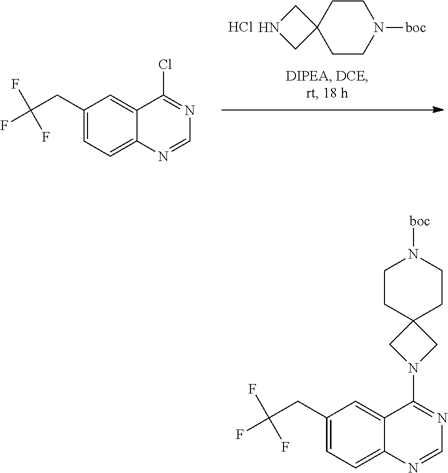

DIPEA (7.9 g, 61 mmol) and tert-butyl 2,7-diazaspiro[3.5]nonane-7-carboxylate hydrochloride (3.2 g, 12.2 mmol) were added to a solution of 4-chloro-6-(2,2,2-trifluoroethyl)quinazoline (P7, 3 g, 12.2 mmol) in dichloroethane (60 ml). The reaction solution was stirred at rt for 18 h (LCMS control). Then product was purified by column chromatography on silica (ethyl acetate-methanol 10:1) to give P8 as a white solid (5.1 g, 96%). ¹H NMR (400 MHZ, DMSO-d₆), δ: 8.47-8.43 (m, 1H), 7.99-7.95 (m, 1H), 7.68 (m, 2H), 4.65-3.96 (m, 4H), 3.96-3.75 (m, 2H), 3.48-3.40 (m, 4H), 2.63-2.48 (m, 4H), 1.86-1.63 (m, 4H), 1.41 (s, 9H).

### Preparation 9 4-(2,7-Diazaspiro[3.5]non-2-yl)-6-(2,2,2-trifluoroethyl)quinazoline (P9)

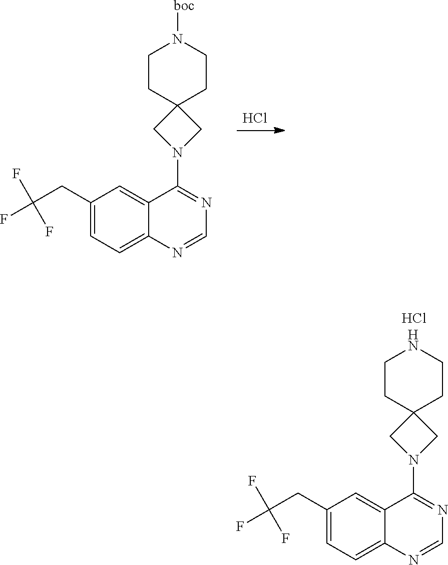

To tert-butyl 2-[6-(2,2,2-trifluoroethyl)quinazolin-4-yl]-2,7-diazaspiro[3.5]nonane-7-carboxylate (P8, 5.1 g, 11.7 mmol) was added dioxane-HCl solution (3 M, 120 ml). The reaction mixture was stirred at rt for 3 h (LCMS control), then evaporated under vacuum to dryness to give 4-(2,7-diazaspiro[3.5]non-2-yl)-6-(2,2,2-trifluoroethyl)quinazoline hydrochloride (P9, 4.4 g, 100%) as a white solid. ¹H NMR (400 MHz, DMSO-d₆), δ: 9.44 (s, 1H), 9.29 (s, 1H), 8.85-8.80 (m, 1H), 8.14-8.08 (m, 1H), 8.06-7.98 (m, 2H), 4.90-4.76 (m, 2H), 4.38-4.24 (m, 2H), 4.03-3.89 (m, 2H), 3.18-2.97 (m, 4H), 2.20-2.02 (m, 4H).

### Synthesis of 5-formyl-4-methyl-1H-indole-2-carbonitrile (P97)

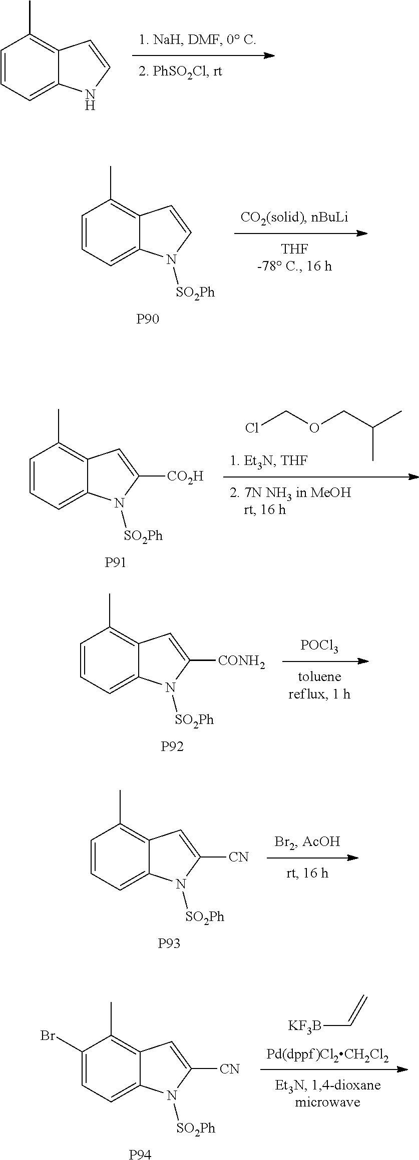

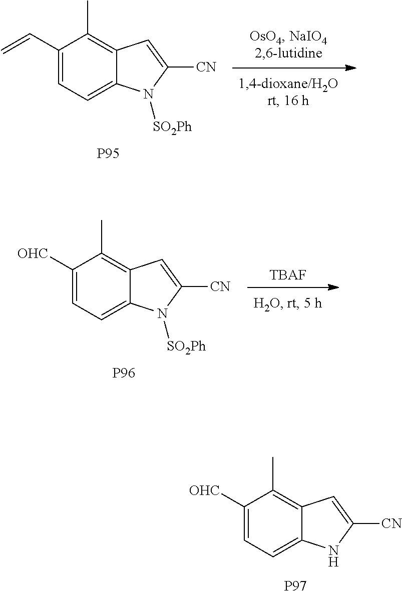

### Preparation 90. 4-Methyl-1-(phenylsulfonyl)-1H-indole (P90)

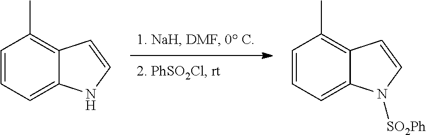

To a solution of 4-methyl-1H-indole (69.0 g, 0.525 mol, 1.0 eq) in anhydrous DMF (600 ml) was added 60% sodium hydride in mineral oil (31.5 g, 0.789 mol, 1.5 eq) at 0° C. After stirred at 0° C. for 1 h, benzenesulfonyl chloride was added to the mixture at 0° C. After the solution was stirred at 0° C. for 16 h, it was quenched with H₂O and extracted with EtOAc. The organic layers were collected, washed with brine, dried over MgSO₄₍ₛ₎, filtered, and concentrated in vacuo to afford P90 (169 g, crude) as a pink solid which was used in next step without further purification. ¹H NMR (400 MHZ, DMSO-d₆), δ: 7.89-7.87 (m, 2H), 7.83 (d, J=8.4 Hz, 1H), 7.56 (d, J=3.6 Hz, 1H), 7.54-7.50 (m, 1H), 7.45-7.41 (m, 2H), 7.23-7.19 (m, 1H), 7.04-7.01 (m, 1H), 6.69 (d, J=3.6 Hz, 1H), 2.47 (s, 3H); LRMS (ESI) [MH]⁺: 272.1.

### Preparation 91. 4-Methyl-1-(phenylsulfonyl)-1H-indole-2-carboxylic acid (P91)

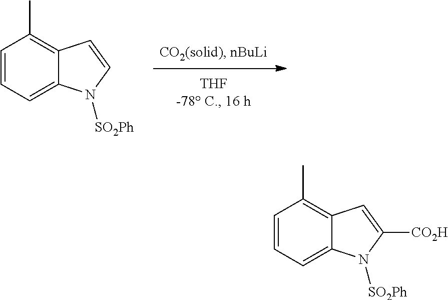

To a solution of P90 (10.0 g, 36.9 mmol, 1.00 eq) in anhydrous THF (100 ml) was added dropwise with a 2.5 M solution of n-butyllithium in hexane (22.1 ml, 55.3 mmol, 1.5 eq) at −78° C. After it was stirred at room temperature for 1 h, the mixture solution was cooled to −78° C. and an excess of CO₂₍ₛ₎ was added. After the solution was stirred at −78° C. for 10 min, the mixture was warmed to rt gradually and stirred for 16 h. The mixture solution was quenched with water and acidified by 1N HCl_{(aq.)}. The solution was extracted with EtOAc, and the organic layers were collected, dried over MgSO₄₍ₛ₎, filtered, and concentrated to afford P91 (10.2 g, 88%) as a purple solid which was used in next step without further purification. ¹H NMR (400 MHZ, DMSO-d₆), δ: 13.58 (s, 1H), 8.01-7.99 (m, 2H), 7.81 (d, J=8.4 Hz, 1H), 7.72-7.68 (m, 1H), 7.62-7.58 (m, 2H), 7.38 (s, 1H), 7.36-7.32 (m, 1H), 7.12-7.10 (m, 1H), 2.43 (s, 3H); LRMS (ESI) [MH]⁺: 316.1.

### Preparation 92. 4-Methyl-1-(phenylsulfonyl)-1H-indole-2-carboxamide (P92)

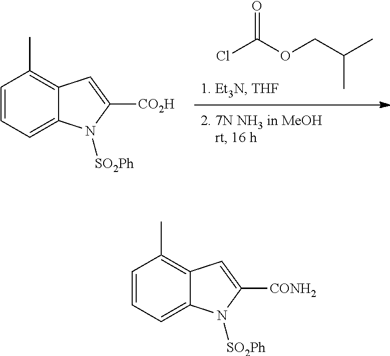

To a stirred solution of P91 (10.2 g, 32.3 mmol, 1.0 eq) in anhydrous THF (170 ml) was added isobutyl carbonochloridate (4.62 ml, 35.6 mmol, 1.1 eq) and TEA (4.96 ml, 35.6 mmol, 1.1 eq) at rt. After the solution was stirred at rt for 1 h, a solution of 7 N ammonia in methanol (23.1 ml, 161 mmol, 5.0 eq) was added. After the mixture solution was stirred at rt for 16 h, it was concentrated and extracted with EtOAc. The organic layers were collected, washed with brine, dried over MgSO₄₍ₛ₎, filtered, and concentrated in vacuo. The residue was purified by flash column chromatography (EtOAc: n-hexane=2:1) to afford P92 (6.3 g, 62%) as a white solid. ¹H NMR (400 MHZ, CDCl₃), δ: 8.20 (br s, 1H), 8.13-8.11 (m, 2H), 7.76 (d, J=8.4 Hz, 1H), 7.70-7.66 (m, 2H), 7.60-7.56 (m, 2H), 7.28-7.24 (m, 1H), 7.08-7.06 (m, 2H), 2.41 (s, 3H); LRMS (ESI) [MH]⁺: 315.1.

### Preparation 93. 4-Methyl-1-(phenylsulfonyl)-1H-indole-2-carbonitrile (P93)

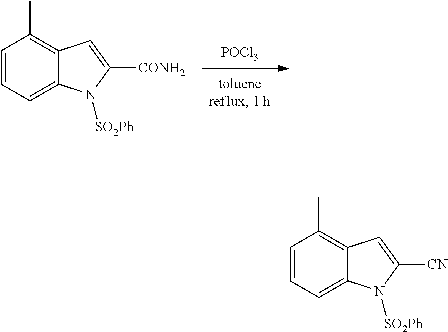

To a solution of P92 (42.9 g, 0.136 mol, 1.0 eq) in toluene (750 ml) was added phosphoryl chloride (44.6 ml, 0.477 mol, 3.5 eq) at rt and stirred at reflux for 1 h. After the solution was cooled to rt, it was quenched with water and neutralized with saturated NaHCO₃(aq.). The mixture solution was extracted with EtOAc. The organic layers were collected, washed with brine, dried over MgSO₄₍ₛ₎, filtered, and concentrated in vacuo. The residue was purified by flash column chromatography (EtOAc: n-hexane=1:3) to give P93 (35.8 g, 88%) as a pale-yellow solid. ¹H NMR (400 MHZ, CDCl₃), δ: 8.11 (s, 1H), 7.98-7.92 (m, 3H), 7.79-7.73 (m, 1H), 7.67-7.62 (m, 2H), 7.53-7.49 (m, 1H), 7.22-7.20 (m, 1H), 2.45 (s, 3H); LRMS (ESI) [MH]⁺: 297.1.

### Preparation 94. 5-Bromo-4-methyl-1-(phenylsulfonyl)-1H-indole-2-carbonitrile (P94)

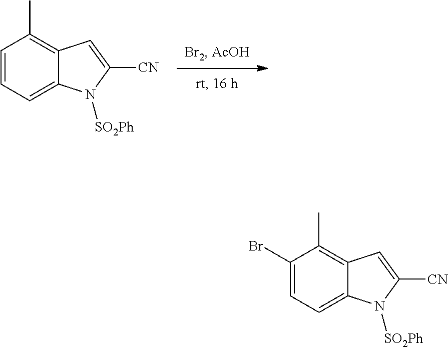

To a solution of P93 (4.17 g, 14.1 mmol, 1.0 eq) in acetic acid (60 ml) was added bromine (1.44 ml, 28.1 mol, 2.0 eq) at rt and the mixture was stirred at rt for 16 h. The mixture solution was diluted with water and extracted with EtOAc. The organic layers were collected, washed with Na₂S₂O_{3(aq.)}, dried over MgSO₄₍ₛ₎, filtered, and concentrated in vacuo. The residue was purified by flash column chromatography (EtOAc: n-hexane=1:5) to give P94 (4.81 g, 91%) as a yellow solid. ¹H NMR (400 MHZ, DMSO-d₆), δ: 8.02-8.00 (m, 2H), 7.95-7.93 (m, 1H), 7.68-7.61 (m, 1H), 7.53-7.49 (m, 2H), 7.39 (s, 1H), 2.51 (s, 3H).

### Preparation 95. 4-Methyl-1-(phenylsulfonyl)-5-vinyl-1H-indole-2-carbonitrile (P95)

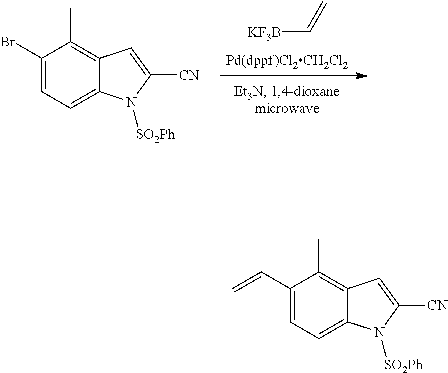

To a solution of P94 (4.00 g, 10.6 mmol, 1.0 eq) in 1,4-dioxane (35 ml) was added potassium vinyltrifluoroborate (2.14 g, 15.9 mmol, 1.5 eq) and TEA (7.42 ml, 53.3 mmol, 5.0 eq) at rt. After the solution was degassed with Argon, 1,1′-bis(diphenylphosphino) ferrocene-palladium (II) dichloride dichloromethane complex (0.870 mg, 1.06 mmol, 0.1 eq) was added. The solution was reacted in a microwave reactor by using a condition of 800 W and 110° C. for 2 h. After the solution was cooled to rt, the mixture was diluted with EtOAc, filtered through a pad of Celite, and concentrated in vacuo. The residue was purified by flash column chromatography (EtOAc: n-hexane=1:6) to give P95 (3.12 g, 91%) as an orange solid. ¹H NMR (400 MHZ, DMSO-d₆), δ: 8.04-8.01 (m, 3H), 7.67 (d, J=8.8 Hz, 1H), 7.63-7.59 (m, 1H), 7.52-7.48 (m, 2H), 7.42 (d, J=1.2 Hz, 1H), 6.99 (dd, J=17.6, 11.2 Hz, 1H), 5.67 (dd, J=17.6, 1.2 Hz, 1H), 5.37 (dd, J=11.2, 1.2 Hz, 1H), 2.45 (s, 3H); LRMS (ESI) [MH]⁺: 323.0.

### Preparation 96. 5-Formyl-4-methyl-1-(phenylsulfonyl)-1H-indole-2-carbonitrile (P96)

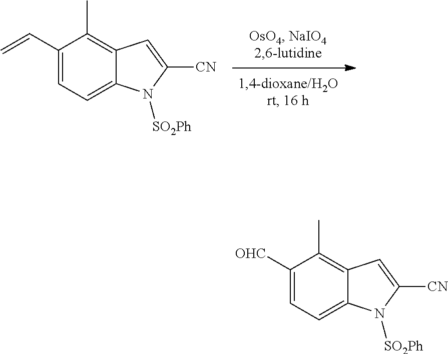

To a solution of P95 (3.12 g, 9.68 mmol, 1.0 eq) in 1,4-dioxane/H₂O (20/5 ml) was added a solution of 2.5%0.0 in t-butanol (1.97 ml, 0.193 mmol, 0.02 eq), sodium periodate (8.28 g, 38.7 mmol, 4.0 eq), 2,6-lutidine (2.25 g, 19.3 mmol, 2.0 eq) at rt and stirred at rt for 16 h. The mixture solution was filtered through a pad of Celite and extracted with EtOAc. The organic layers were collected, washed with brine, dried over MgSO₄₍ₛ₎, filtered, and concentrated in vacuo. The residue was purified by flash column chromatography (EtOAc: n-hexane=1:2) to give P96 (1.46 g, 46%) as a yellow solid. ¹H NMR (400 MHZ, DMSO-d₆), δ: 10.38 (s, 1H), 8.20 (d, J=8.8 Hz, 1H), 8.07-8.05 (m, 2H), 8.01 (d, J=8.8 Hz, 1H), 7.68-7.63 (m, 1H), 7.56-7.52 (m, 3H), 2.82 (s, 3H); LRMS (ESI) [MH]⁺: 325.1.

### Preparation 97. 5-Formyl-4-methyl-1H-indole-2-carbonitrile (P97)

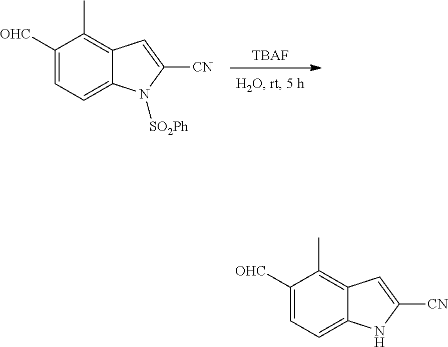

To a solution of P96 (1.46 g, 4.50 mmol, 1.0 eq) in THF (22 ml) was added 1 M tetra-n-butylammonium fluoride in THF (5.40 ml, 5.40 mmol, 1.2 eq) and stirred at rt for 5 h. The mixture solution was filtered through a pad of Celite and extracted with EtOAc. The organic layers were collected, washed with brine, dried over MgSO₄₍ₛ₎, filtered, and concentrated in vacuo to give a crude solid. The crude solid was washed with methanol and collected by filtration to give P97 (672 mg, 81%) as a pale-yellow solid. ¹H NMR (400 MHz, DMSO-d₆), δ: 12.93 (br. s, 1H), 10.35 (s, 1H), 7.78-7.75 (m, 2H), 7.42 (d, J=8.4 Hz, 1H), 2.85 (s, 3H); LRMS (ESI) [MH]⁺: 185.1.

### Synthesis of 5-formyl-4-methyl-1-[[(2R)-5-oxomorpholin-2-yl]methyl]indole-2-carbonitrile (P176)

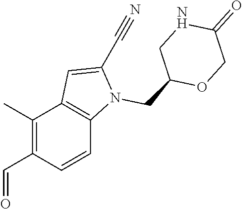 [SMILES: Cc1c(C=O)ccc2c1cc(C#N)n2C[C@H]1CNC(=O)CO1]

### Preparation 174. 2-Chloro-N-[(2R)-2,3-dihydroxypropyl] acetamide (P174)

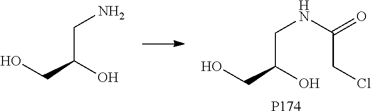 [SMILES: "P174" ; NC[C@@H](O)CO ; O=C(CCl)NC[C@@H](O)CO]

To a solution of (2R)-3-aminopropane-1,2-diol (10 g, 0.110 mol) in a mixture of MeCN (330 ml) and MeOH (63 ml) triethylamine (13.32 g, 0.132 mol) was added. Chloroacetyl chloride (13.77 g, 0.122 mol) was then added dropwise with stirring at temperature −20° C. during 1 h under argon. The reaction mixture was allowed to reach rt and stirred overnight. The reaction mixture was concentrated to dryness, shaken with dry ethyl acetate (0.5 L), solution filtered and dried. Ethyl acetate was evaporated to give pure 2-chloro-N-[(2R)-2,3-dihydroxypropyl] acetamide P174 (9.2 g, 50%), as a beige oil. ¹H NMR (400 MHZ, DMSO-d₆), δ: 8.02 (s, 1H), 4.27 (s, 1H), 4.48 (s, 1H), 4.06 (s, 2H), 3.53 (m, 1H), 3.31 (m, 3H), 3.03 (m, 1H).

### Preparation 175. (6R)-6-(Hydroxymethyl) morpholin-3-one (P175)

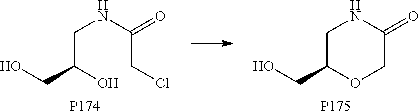 [SMILES: "P174" ; "P175" ; O=C(CCl)NC[C@@H](O)CO ; O=C1CO[C@@H](CO)CN1]

Potassium tert-butoxide (15.4 g, 0.138 mol) was dissolved in tert-BuOH (100 ml), then solution of 2-chloro-N-[(2R)-2,3-dihydroxypropyl] acetamide (P174, 9.2 g, 0.055 mol) in tert-BuOH (220 ml) was added with stirring at temperature 30° C. over 1 h under argon. The reaction mixture was stirred for 1 h, then MeOH (50 ml) and water (3 ml) were added and the reaction was stirred for an additional 15 min. The crude was concentrated and purified by flash chromatography on silica gel with MeOH/EtOAc (30:70) to provide (6R)-6-, (hydroxymethyl) morpholin-3-one (P175, 3.4 g, 47%) as a white solid. ¹H NMR (400 MHz, DMSO-d₆), δ: 7.93 (s, 1H), 4.85 (t, J=5.5 Hz, 1H), 4.00 (m, 2H), 3.65 (m, 1H), 3.45 (m, 2H) 3.18 (m, 1H), 3.07 (m, 1H).

### Preparation 176. 5-Formyl-4-methyl-1-{[(2R)-5-oxomorpholin-2-yl]methyl}-1H-indole-2-carbonitrile (P176)

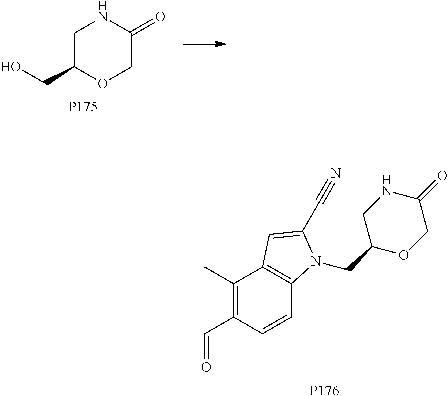 [SMILES: "P175" ; "P176" ; O=C1CO[C@@H](CO)CN1 ; Cc1c(C=O)ccc2c1cc(C#N)n2C[C@H]1CNC(=O)CO1]

To a solution of (6R)-6-(hydroxymethyl) morpholin-3-one (P175, 1.07 g, 8.15 mmol) in DMAA (20 ml) DIPEA (1.6 g, 12.25 mmol) and MsCl (1.21 g, 10.6 mmol) were added. The mixture was stirred at 50° C. for 45 min. Then 2-cyano-4-methyl-5-formylindole (P97, 0.5 g, 2.72 mmol) and potassium carbonate (3 g, 22 mmol) were added, and the mixture was stirred at 100° C. for additional 18 h and quenched with water (100 ml). After extraction with ethyl acetate (3×100 ml) and evaporation to dryness obtained residue was washed with dry ethyl acetate (150 ml), filtered, and dried to give 5-formyl-4-methyl-1-{[(2R)-5-oxomorpholin-2-yl]methyl}-1H-indole-2-carbonitrile (P176, 245 mg, 30%). ¹H NMR (400 MHZ, DMSO-d₆), δ: 12.37 (s, 1H), 8.03 (m, 1H), 7.84 (m, 2H), 7.69 (d, J=12.2 Hz, 1H), 4.63 (m, 1H), 4.44 (m, 1H), 4.05 (m, 1H), 3.90 (m, 2H), 3.40 (m, 1H), 3.17 (m, 1H), 2.84 (s, 3H).

### Synthesis of 5-formyl-4-methyl-1-[(2S)-5-oxomorpholin-2-yl]methyl]indole-2-carbonitrile (P177)

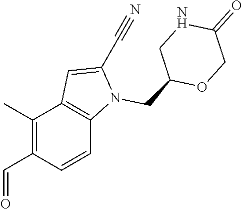 [SMILES: Cc1c(C=O)ccc2c1cc(C#N)n2C[C@H]1CNC(=O)CO1]

This compound P177 was obtained using procedures described above for the synthesis of P176 (Preparations 174-176), using (2S)-3-aminopropane-1,2-diol instead of (2R)-3-aminopropane-1,2-diol at the first step.

### Synthesis of the Compounds

### Example 45. 4-Methyl-1-{[(2R)-5-oxomorpholin-2-yl]methyl}-5-({2-[6-(2,2,2-trifluoroethyl)quinazolin-4-yl]-2,7-diazaspiro[3.5]non-7-yl}methyl)-1H-indole-2-carbonitrile (Compound 101)

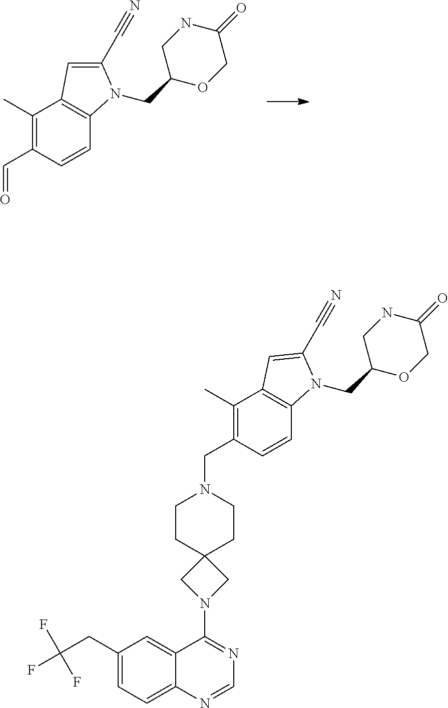 [SMILES: Cc1c(C=O)ccc2c1cc(C#N)n2C[C@H]1CNC(=O)CO1 ; Cc1c(CN2CCC3(CC2)CN(c2ncnc4ccc(CC(F)(F)F)cc24)C3)ccc2c1cc(C#N)n2C[C@H]1CNC(=O)CO1]

A mixture of 5-formyl-4-methyl-1-{[(2R)-5-oxomorpholin-2-yl]methyl}-1H-indole-2-carbonitrile (P176, 177 mg, 0.60 mmol), 4-(2,7-diazaspiro[3.5]non-2-yl)-6-(2,2,2-trifluoroethyl)quinazoline (P9, 200 mg, 0.6 mmol), DIPEA (384 mg, 2.98 mmol) and STAB (631 mg, 2.98 mmol) in DCM (30 ml) was stirred for 24 h, at rt (LCMS control), then with stirring a water solution of sodium bicarbonate was added. The mixture was stirred 30 min, water phase extracted with DCM (2×20 ml), evaporated to dryness. Residue was refluxed with MeCN (30 ml) for 2 h, cooled, filtered, washed with MeCN (10 ml), and dried to yield pure 4-methyl-1-{[(2R)-5-oxomorpholin-2-yl]methyl}-5-({2-[6-(2,2,2-trifluoroethyl)quinazolin-4-yl]-2,7-diazaspiro[3.5]non-7-yl}methyl)-1H-indole-2-carbonitrile (Compound 101, 174 mg, 47%). ¹H NMR (400 MHZ, DMSO-d₆), δ: 8.46 (s, 1H), 7.99 (m, 2H), 7.73 (m, 2H), 7.52 (m, 1H), 7.46 (d, J=5.6 Hz, 1H), 7.31 (d, J-4.8 Hz, 1H), 4.54 (m, 1H), 4.20 (m, 2H), 4.05 (m, 1H), 3.90 (m, 4H), 3.52 (m, 2H), 3.35 (m, 1H), 3.17 (m, 1H), 2.39 (m, 2H), 1.79 (m, 4H). LCMS (ESI) [MH]⁺: 618.

### Example 46. 4-Methyl-1-{[(2S)-5-oxomorpholin-2-yl]methyl}-5-({2-[6-(2,2,2-trifluoroethyl)quinazolin-4-yl]-2,7-diazaspiro[3.5]non-7-yl}methyl)-1H-indole-2-carbonitrile (Compound 102)

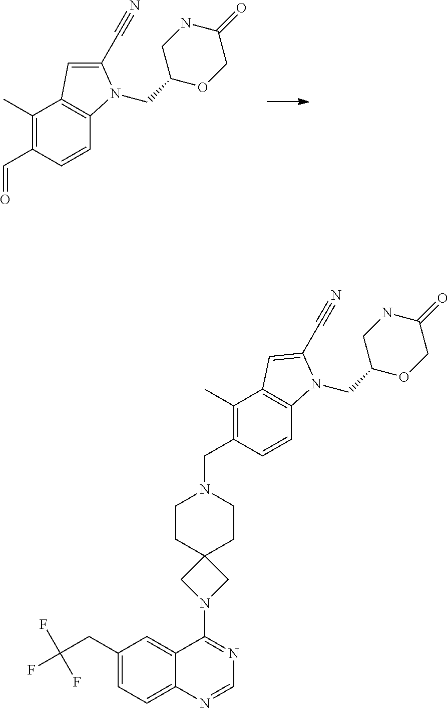 [SMILES: Cc1c(C=O)ccc2c1cc(C#N)n2C[C@@H]1CNC(=O)CO1 ; Cc1c(CN2CCC3(CC2)CN(c2ncnc4ccc(CC(F)(F)F)cc24)C3)ccc2c1cc(C#N)n2C[C@@H]1CNC(=O)CO1]

Compound was prepared using procedure described in the Example 45 and 5-formyl-4-methyl-1-{[(2S)-5-oxomorpholin-2-yl]methyl}-1/-indole-2-carbonitrile P177 instead of 5-formyl-4-methyl-1-{[(2R)-5-oxomorpholin-2-yl]methyl}-1H-indole-2-carbonitrile P176. Compound 102 was obtained with yield 49%. ¹H NMR (400 MHZ, DMSO-d₆), δ: 8.46 (s, 1H), 7.99 (m, 2H), 7.73 (m, 2H), 7.52 (m, 1H), 7.46 (d, J=5.6 Hz, 1H), 7.31 (d, J=4.8 Hz, 1H), 4.54 (m, 1H), 4.20 (m, 2H), 4.05 (m, 1H), 3.90 (m, 4H), 3.52 (m, 2H), 3.35 (m, 1H), 3.17 (m, 1H), 2.39 (m, 2H), 1.79 (m, 4H). LCMS (ESI) [MH] *: 618.

### Biological Assays

### Example A. Primary Assay Used to Determine Potency of MEN1 Activity Inhibition

Compound activity was determined using recombinant MEN1 protein (Creativebiomart, Cat #MEN1-35H) and a custom fluorescein-labeled MLL4-43 peptide (Eton Bioscience Inc.). Interaction between MEN1 and MLL4-43 in the presence of compounds was determined by fluorescence polarization assay using a Microplate Reader ClarioStar Plus. The reaction was carried out in assay buffer (50 mM TRIS-HCl PH 7.4-7.6, 50 mM NaCl, 1 mM DTT, 0.1 mg/ml BSA). The compounds were dispensed on a 384 well Diamond Well Plate (Axigen, Cat #P-384-120 SQ-C-S) using the Biomek FX liquid handling system at 100×solutions of compounds in DMSO. 2×MEN1 mix (final concentration of MEN1 10 nM) was prepared in Assay buffer and 10 μl of mixture per well was added into 384w white Reaction plate with NBS (Corning, Cat #4513). 10 μl of Assay buffer w/o MEN1 was used for negative control. Plates were centrifuged for 1 min at 100 g. Next step the Compounds were added to Reaction plate using Biomek station via following steps: 3 μl of 100× compounds (in DMSO) were mixed thoroughly with 27 μl Assay Buffer, then 2 μl of this mixture was added to Reaction plate with 10 μl of MEN1 mix. Plates were centrifuged for 1 min at 100 g and incubated for 20 min at room temperature. Next 8 μL of MLL4-43 peptide per well was added to final concentration of MLL 0.5 nM. Plates were incubated for 1 hour at room temperature. Then fluorescence polarization was measured using Microplate Reader. The results of this assay are shown in the Table A. The values of EC₅₀ shown as a letters A-E, where: A≤0.075 μM; 0.075 μM<B≤0.5 μM; 0.5 μM<C≤1 μM; 1<D≤5 μM; E>5.

TABLE A
MEN1 Activity Inhibition Assays

| Compound Number | EC₅₀, uM* |
|---|---|
| 101 | A |
| 102 | A |

*EC₅₀: Half maximal effective concentration: the concentration of a compound which induces a response halfway between the baseline and maximum after a specific exposure time; EC₅₀: A ≤ 0.075 μM; 0.075 μM < B ≤ 0.5 μM; 0.5 μM < C ≤ 1 μM; 1 < D ≤ 5 μM; E > 5

### Example B. Cellular Growth Inhibition Assay

HEK293 (Institute of Cytology Russian Academy of Science), MV4-11 (ATCC, CRL-9591), MOLM-13 (AcceGen, ABC-TC517S) were seeded at a density of 500 cells per well (HEK293) and 2000 cells per well (MV4-11, MOLM-13) in a 384-well clear bottom plate (Greiner Cat #781090) in 45 μl total volume of DMEM (PanEco, Cat #C420, Russia) or RPMI (PanEco, Cat #C330, Russia) with 10% FBS (HyClone Cat #SV30160.03). HEK293 were allowed to adhere overnight at 37° C., 5% CO₂. 500× compounds solutions in DMSO (Sigma Cat #D2650) were prepared into Cmpnds plate (Diamond Well Plate, Axigen, Cat #P-384-120 SQ-C-S) and DMSO only control was included. 1 μl of 500× compounds (Cmpnds plate) was added to 49 μl of culture medium into Dilution plate (Diamond Well Plate, Axigen, Cat #P-384-120 SQ-C-S), mixed and then 5 μl of 10× compounds solutions were transferred to cells followed by centrifugation at 100 g for 1 min. Final DMSO concentration was 0.2%. After 3 days of incubation, 10 μl of 1× compounds were added to cells. After 7 days of incubation, 12 μl of CellTiter-Glo (Promega, CAT #G7572) were added to the cells, plate was centrifuged at 100 g for 1 min and luminescence signal was measured using Microplate Reader (CLARIOStar). The results of these assays are shown in the Tables B1, B2, and B3.

TABLE B1
MV4-11 Cellular Growth Inhibition Assay

| Compound Number | MV4-11, CC₅₀,* μM |
|---|---|
| 101 | B |
| 102 | A |

*CC₅₀: Cytoxic Concentration: the extract concentration that reduced the cell viability by 50% when compared to untreated controls; MV4-11 CC₅₀: A ≤ 0.1 μM; 0.1 μM < B ≤ 0.5 μM; 0.5 μM < C ≤ 1 μM; 1 < D ≤ 5 μM; E > 5

TABLE B2
MOLM-13 Cellular Growth Inhibition Assay

| Compound Number | MOLM-13 CC₅₀,^{a} μM |
|---|---|
| 101 | D |
| 102 | B |

* CC₅₀: Cytotoxic Concentration: the extract concentration that reduced the cell viability by 50% when compared to untreated controls; MOLM-13 CC₅₀: A ≤ 0.1 μM; 0.1 μM < B ≤ 0.5 μM; 0.5 μM < C ≤ 1 μM; 1 < D ≤ 5 μM; E > 5

TABLE B3
HEK293 Cellular Growth Inhibition Assay

| Compound Number | HEK293, CC₅₀,* μM |
|---|---|
| 101 | B |
| 102 | A |

*CC₅₀: Cytotoxic Concentration: the extract concentration that reduced the cell viability by 50% when compared to untreated controls; HEK293 CC₅₀: A ≤ 2 μM; 2 μM < B ≤ 5 μM; 5 μM < C ≤ 10 μM; 10 < D ≤ 20 μM; E > 20

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound selected from: 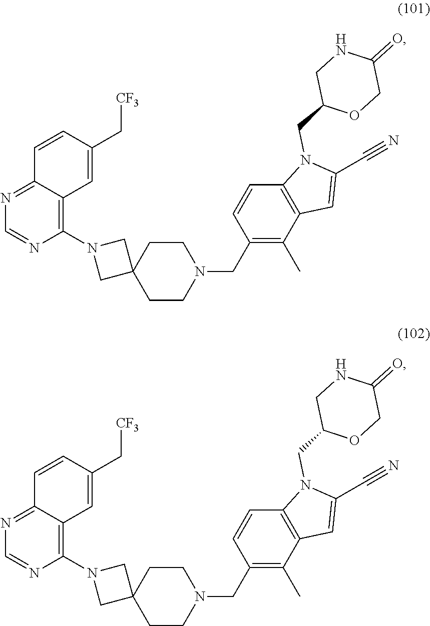 [SMILES: Cc1c(CN2CCC3(CC2)CN(c2ncnc4ccc(CC(F)(F)F)cc24)C3)ccc2c1cc(C#N)n2C[C@H]1CNC(=O)CO1 ; Cc1c(CN2CCC3(CC2)CN(c2ncnc4ccc(CC(F)(F)F)cc24)C3)ccc2c1cc(C#N)n2C[C@@H]1CNC(=O)CO1]
or a pharmaceutically acceptable salt, prodrug, stereoisomer, solvate, or tautomer thereof.

2. A pharmaceutical composition comprising the compound of any one of claim 1 or a pharmaceutically acceptable salt, stereoisomer, solvate, or tautomer thereof, and a pharmaceutically acceptable carrier.

3. The pharmaceutical composition of claim 2, further comprising one or more additional pharmaceutically active agents.

4. A method of inhibiting the interaction of menin and MLL in a cell, comprising contacting the cell with a compound of claim 1.

5. The method of claim 4, wherein the contacting is in vitro or in vivo.

6. A method for the treatment or prevention of a disease or disorder associated with the interaction of menin and MLL comprising administering to a subject in need thereof a compound of claim 1.

7. The method of claim 6, wherein the disease or disorder is selected from the group consisting of a leukemia, hematologic malignancy, solid tumor cancer, prostate cancer, breast cancer, liver cancer, brain tumor, and diabetes.

8. The method of claim 7, wherein the leukemia is selected from the group consisting of AML, ALL, Mixed Lineage Leukemia, and a leukemia with Partial Tandem Duplications of MLL.

9. The method of any one of claims 6-8, wherein the subject is a mammal.

10. The method of claim 9, wherein the subject is a human.
